(19) United States
(12) Patent Application Publication
(10) Pub. No.: US 2012/0264784 A1
(43) Pub. Date: Oct. 18, 2012

(54) METHODS OF TREATING ELEVATIONS IN MTOR SIGNALING

(76) Inventors: Mark F. Bear, Dartmouth, MA (US); Benjamin David Auerbach, Somerville, MA (US)

(21) Appl. No.: 13/455,679

(22) Filed: Apr. 25, 2012

Related U.S. Application Data
(63) Continuation of application No. PCT/US2010/055268, filed on Nov. 3, 2010.
(60) Provisional application No. 61/258,453, filed on Nov. 5, 2009.
(60) Provisional application No. 61/260,769, filed on Nov. 12, 2009.
(60) Provisional application No. 61/359,648, filed on Jun. 29, 2010.
(60) Provisional application No. 61/359,604, filed on Jun. 29, 2010.
(60) Provisional application No. 61/387,649, filed on Sep. 29, 2010.

(51) Int. Cl. A61K 31/454; A61K 31/195; A61K 31/15; A61K 31/4035; A61P 25/08; A61K 31/40; A61K 31/352; A61K 31/27; A61P 25/00; A61K 31/4245; A61K 31/415

(52) U.S. Cl. 514/326; 514/364; 514/567; 514/639; 514/417; 514/407; 514/424; 514/454; 514/487

(57) ABSTRACT

Subjects having elevated signaling of a mammalian target of rapamycin (mTOR) are treated with compositions that include at least one compound that activates a Group 1 mGluR. In an embodiment, the subject has tuberous sclerosis complex (TSC). In an embodiment, the compound is a Group 1 mGluR agonist. In another embodiment, the compound is a Group 1 mGluR positive allosteric modulator.

### RELATED APPLICATIONS

This application is a continuation of and claims priority to International Application No. PCT/US2010/055268, which designated the United States and was filed on Nov. 3, 2010, published in English, which claims the benefit of U.S. Provisional Application No. 61/258,453, filed Nov. 5, 2009; U.S. Provisional Application No. 61/260,769, filed Nov. 12, 2009; U.S. Provisional Application No. 61/359,648, filed Jun. 29, 2010; U.S. Provisional Application No. 61/359,604, filed Jun. 29, 2010 and U.S. Provisional Application No. 61/387,649, filed Sep. 29, 2010. The entire teachings of the above applications are incorporated herein by reference.

### BACKGROUND OF THE INVENTION

Proper neuronal cell signaling is critical to maintaining the integrity of synapses for normal functioning, including behavioral and cognitive functioning. Mutations in the mammalian target of rapamycin (mTOR) signaling pathway can result in altered neuronal cell signaling, including the formation of tumors, altered behaviors and impairments in cognitive processes. For example, elevations in mTOR signaling occur in Tuberous Sclerosis Complex (TSC).

Humans with mutations in mTOR signaling, such as humans with TSC, can have developmental delays in cognitive processing, mental retardation, anxiety, autism and seizures, which can affect day-to-day functioning by impairing learning, memory, speech, social skills and behavior. Currently, available treatment regimens for humans with mutations in mTOR signaling include surgical removal of tumors, behavioral modifications, cognitive behavioral therapy and treatment with anti-seizure medications. However, such treatments frequently are not effective, may produce undesirable side-effects with long term use and are not specifically directed to treating cognitive impairments associated with mutations in mTOR. Thus, there is a need to develop new, improved and effective methods to treat conditions associated with mutations in mTOR signaling.

### SUMMARY OF THE INVENTION

The invention is generally directed to methods of treating subjects having an elevation in mammalian target of rapamycin (mTOR) signaling.

In an embodiment, the invention is a method of treating a subject having elevated signaling of a mammalian target of rapamycin (mTOR), comprising the step of administering to the subject a composition that includes at least one compound that activates Group I mGluR signaling.

Advantages of the claimed methods can include, for example, treatment of subjects in a manner that can improve efficacy and quality of life and have the potential for minimal side effects, thereby improving tolerability for use over a relatively long periods of time. The methods of the invention may provide an effective means to treat cognitive, learning, social, behavioral, language, communication and development impairments in a subject having elevated signaling of mTOR by normalizing synaptic function.

### BRIEF DESCRIPTION OF THE FIGURES

FIG. 1 depicts a model of regulation and function of TSC1/2 complex by growth factors, such as insulin or BDNF.

FIG. 2 depicts normal mTOR cell signaling, elevated mTOR cell signaling and synaptic function.

FIGS. 3A, 3B, 3C, 3D and 3E depict protein synthesis-dependent component of mGluR-LTD that is absent in Tsc2^{+/-} mice.

FIGS. 4A, 4B and 4C depict basal synaptic transmission in the CA1 hippocampal region and normal NMDAR-LTD in wildtype and Tsc2^{+/-} mice.

FIGS. 5A, 5B, 5C, 5D and 5E depict excessive mTOR activity suppresses a protein-synthesis-dependent component of mGluR-LTD that can be overcome by augmenting mGluR5.

FIGS. 6A and 6B depict positive modulation of mGluR 5 reverses context discrimination deficit in Tsc2^{+/-} mice.

FIG. 7 depicts mGluR signaling, altered mTOR signaling and the effects of mGluR5 PAM.

FIG. 8 depicts mGluR signaling, altered mTOR signaling and the effects of mGluR5 PAM.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

In one embodiment, the invention is a method of treating a subject having elevated signaling of a mammalian target of rapamycin (mTOR), comprising the step of administering to the subject a composition that includes at least one compound that activates Group I mGluR signaling, such as activation of Group 1 mGluR5 signaling or Group 1 mGluR1 signaling.

“Elevated,” as used herein in reference to mTOR signaling, means an increase in mTOR signaling compared to a normal level of mTOR signaling in a cell or a subject.

mTOR is a serine/threonine protein kinase that plays a key role in regulating cell proliferation. mTOR initiates signaling that can control activation of translational machinery, resulting in the translation of specific mRNAs. mTOR is regulated by the PI3 kinase/Akt signaling pathway and through autophosphorylation. When mTOR is not properly regulated (e.g., elevated activity) tumors can develop, as in TSC.

mTOR is present in two distinct complexes. mTOR complex 1 (mTORC1) includes mTOR, Raptor and GβL (mLST8) and is inhibited by rapamycin. mTORC1 integrates multiple signals for the availability of growth factors, nutrients or energy to promote cellular growth when conditions are favorable or catabolic processes during stress or when growth conditions are unfavorable. Growth factors, such as insulin like growth factor (ILGF), signal mTORC1 by activating Akt or ERK1/2, which, in turn, inactivates, for example, TSC2 (i.e., the protein encoded by the tuberous sclerosis complex 2 (TSC2) gene) to prevent TSC2 inhibition of mTORC1. Alternatively, low ATP levels lead to the AMPK-dependent activation of TSC2 to reduce mTORC1 signaling. Amino acid availability is signaled to mTORC1 by a pathway involving the Rag proteins. Active mTORC1 has several downstream biological effects including translation of mRNA by the phosphorylation of downstream targets, such as 4E-BP1 and p70 S6 Kinase, suppression of autophagy, ribosome biogenesis and activation of transcription leading to mitochondrial metabolism or adipogenesis.

The mTOR complex 2 (mTORC2) includes mTOR, Rictor, GβL and Sin1. mTORC2 promotes cell survival by activating Akt. mTORC2 also regulates cytoskeletal dynamics by activating at least one member selected from the group consisting of PKCα and Rho GTPase. Aberrant mTOR signaling is involved in many disease states including cancer, cardiovascular disease and metabolic disorders.

Elevated mTOR signaling can be assessed by employing commercially available kits, such as AlphaScreen® SureFire® p-eIF4E Ser209 kit; AlphaScreen® SureFire® Phospho-4EBP 1 (Thr37/Thr46) Kit; AlphaScreen® SureFire® Phospho-4EBP 1 (Thr70) Assay Kit; AlphaScreen® SureFire® Phospho-mTOR (Ser2448) Assay Kit; AlphaScreen® SureFire® Phospho-mTOR (Ser2481) Assay Kit; AlphaScreen® SureFire® Phospho-p70 S6K (Thr229) Assay Kit; AlphaScreen® SureFire® Phospho-p70 S6K (Thr389) Assay Kit; AlphaScreen® SureFire® Phospho-p70 S6K (Thr421/Ser424) Assay Kit; AlphaScreen® SureFire® Phospho-S6 RP (Ser235/Ser236) Assay Kit and AlphaScreen® SureFire® Phospho-S6 RP (Ser240/Ser244) Assay (PerkinElmer), which measure mTOR signaling by cellular immunoassay assays that measure levels of phosphorylated cellular protein targets involved in mTOR signaling pathways.

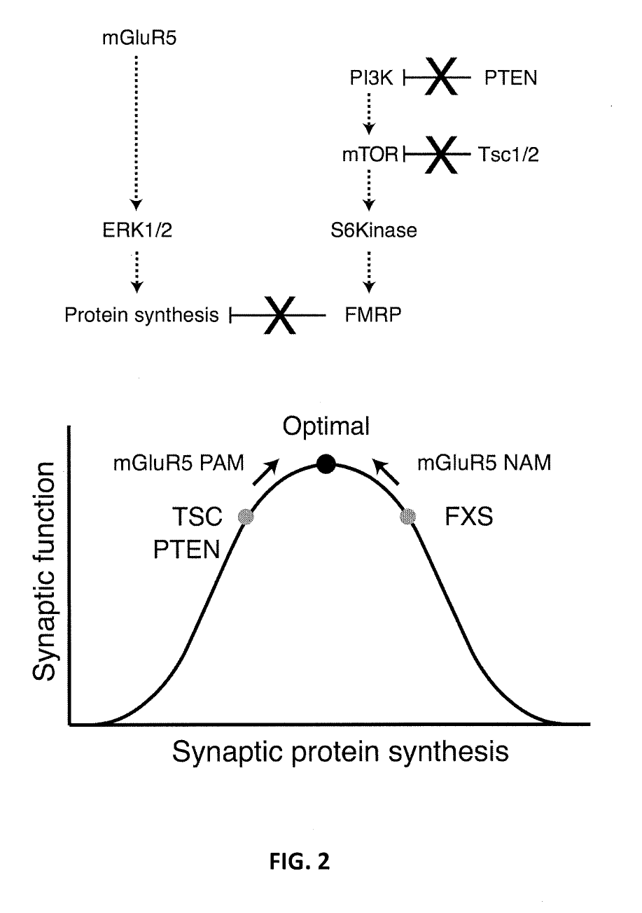

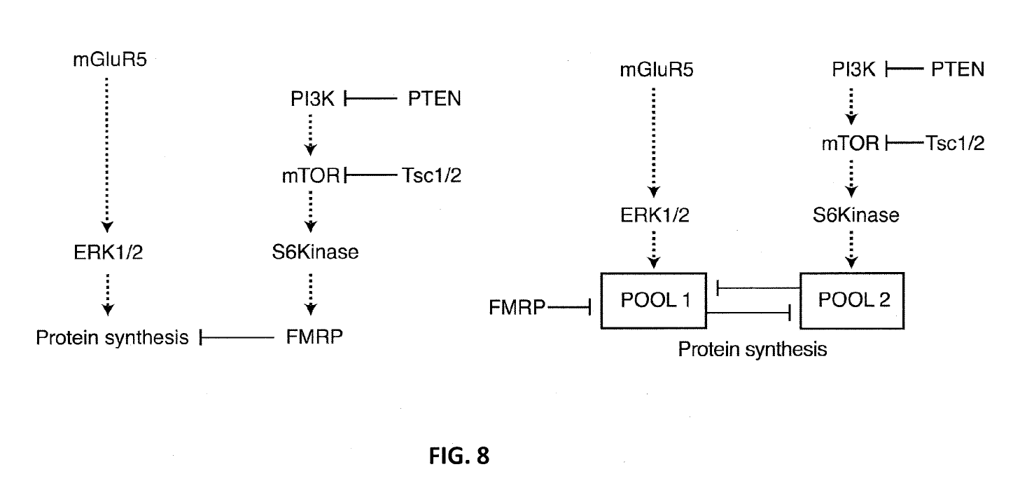

An elevation in mTOR signaling can be, for example, a consequence of removal of inhibition of mTOR in the absence of at least one protein product of at least one member selected from the group consisting of TSC1, TSC2 and PTEN, which normally inhibit mTOR activity in cells (See FIGS. 2 and 8). In the absence of, for example, TSC1, TSC2, PTEN, or mutations in the genes encoding these proteins, mTOR signaling is elevated and, in turn, ultimately activates S6Kinase. In neurons, activation of S6Kinase, consequent to an elevation in mTOR cell signaling, may result in phosphorylation of fragile X mental retardation protein (FMRP), which, in turn, suppresses protein synthesis in neurons, specifically suppressing protein synthesis in dendrites (postsynaptic neurons), as depicted in FIGS. 2, 7 and 8. Suppression of protein synthesis in postsynaptic neurons can be assessed by measuring synaptic long-term depression (LTD) in response to activation of Group I mGluRs. Phosphorylation of FMRP, consequent to, directly or indirectly, an elevation in mTOR signaling, may suppress postsynaptic protein synthesis, which can result in decreased LTD, as depicted in FIGS. 2, 7, and 8.

In an embodiment, the subject treated by the methods of the invention that has elevated signaling of mTOR has tuberous sclerosis complex (TSC). The administration of a Group I mGluR agonist normalizes synaptic signaling, thereby treating the subject.

TSC is caused by a heterozygous, rare, single gene mutation (inactivating mutation) in either the TSC1 or TSC2 gene. The TSC1 gene is located on chromosome 9 and is referred to as the hamartin gene. The TSC2 gene is located on chromosome 16 and is referred to as the tuberin gene. The protein products of the TSC1 and TSC2 genes form a complex involved in tumor suppression in many tissue types. When one or both of these genes are defective in TSC, tumors are not suppressed, resulting in benign tumors (hemartomas) in several organs, including the brain. TSC affects about 1 in every 6000 individuals. Up to about 90% of humans with TSC suffer from epilepsy and about 50% from mental retardation (intelligence quotient about <70) (Joinson, C., et al., Psychol. Med. 33:335-344 (2003)). One skilled in the art would be able to assess whether a subject has TSC using established clinical criteria. For example, major features that are diagnostic of TSC include facial angiofibromas or forehead plaque; non-traumatic ungual or periungual fibroma; hypomelanotic macules (more than three); shagreen patch (connective tissue nevus); multiple retinal nodular hamartomas; cortical tubera; subependymal nodule; subependymal giant cell astrocytoma; cardiac rhabdomyoma, single or multiple; lymphangiomyomatosisb and renal angiomyolipomab. Minor features that are diagnostic of TSC include multiple randomly distributed pits in dental enamel; hamartomatous rectal polyps; bone cysts; cerebral white matter migration lines; gingival fibromas; non-renal hamartoma; retinal achromic patch; “Confetti” skin lesions; and multiple renal cysts. A clinical diagnosis of definitive TSC includes either two major features or one major feature with two minor features.

Molecular genetic testing is commercially available for confirming a diagnosis of TSC when a clinical criteria indicates a subject has TSC. Genetic testing detects a mutation in the TSC1 gene and/or the TSC2 gene.

The protein products of TSC1 and TSC2 form a complex that functions as guanosine triphosphate activating proteins (GAP) that inhibit mammalian target of rapamycin (mTOR) signaling. mTOR is a member of the phosphoinositide kinase-related kinase (PIKK) family that phosphorylates serine and threonine residues of proteins, including S6 kinase. Phosphorylation of S6 kinase, in turn, modifies protein synthesis in cells, including neurons.

In neurons, in the absence of TSC1 and/or TSC2, suppression of mTOR activity is removed. Activated mTOR, in turn, may result in the phosphorylation of S6 kinase. As described herein, in a well-recognized model of TSC, the Tsc2^{+/-} mouse, there is decreased synaptic protein synthesis and decreased long-term depression (LTD).

LTD is a well-recognized indicator of synaptic strength and a functional readout of mGluR-dependent protein synthesis (Huber, K. M., et al., Science 288:1254-1257 (2000)). The regulation of protein synthesis is critical for proper functioning in many organs and tissues, including the brain. Long-term maintenance of synaptic plasticity requires the synthesis of new proteins. Persistent modification of synaptic strengths may be the neural basis of learning and memory. Improper regulation of synaptic protein synthesis leads to altered synaptic plasticity and adversely affects learning, memory and cognition. An elevation in mTOR cell signaling can decrease postsynaptic protein synthesis (e.g., decreased LTD), as described herein. Decreased synaptic protein synthesis may contribute to the learning and cognitive deficiencies observed in individuals with elevation in mTOR signaling. The methods of the invention normalize synaptic protein synthesis by increasing by mGluR activation protein synthesis in neurons of subjects with an elevation in mTOR signaling, such as a subject with TSC, to thereby treat the subject, including cognitive and learning deficits associated with an elevation in mTOR signaling.

Central nervous system dysfunction is a defining factor in TSC, with the some of the most common clinical features being mental retardation, epilepsy, autism, anxiety and mood disorders (Prather, P., et al., J Child Neurol 19, 666-74 (2004)). The two most prevalent disruptions associated with TSC are seizures and mental retardation, seen in about 90% and about 50% of patients, respectively (Shepherd, C. W., et al., AJNR Am J Neuroradiol 16, 149-55 (1995)). The third major feature of the disorder is a high occurrence of autism, with autistic features present in 25-60% of TSC patients and TSC accounting for 1-4% of the autistic population (Wiznitzer, M., et al., J Child Neurol 19, 675-9 (2004)). Currently, there is no cure for TSC and no treatment directed at the cognitive impairments associated with the disorder.

The role of cortical tubers in the electrophysiological and behavioral impairments in TSC is not clear. Some studies show that tuber levels and instances of seizures are correlated (Goodman, M., et al., J Child Neurol 12, 85-90 (1997)). However, several studies have failed to show a similar relationship (Bolton, P. F., et al., Brain 125, 1247-55 (2002); and Walz, N. C., et al., J Child Neurol 17, 830-2 (2002)). Furthermore, recordings of epilepsy resection tissue from TSC patients indicate that the excitatory/inhibitory balance is altered in a direction that favors seizure generation in the TSC brain outside of the cortical tubers (Wang, Y., et al., Ann Neurol 61, 139-52 (2007); and Goorden, S. M., et al., Ann Neurol 62, 648-55 (2007)). New evidence from mouse models suggests that the cognitive deficits seen in TSC may be dissociated from the neuroanatomical pathology (Kaufmann, R., et al., J Child Neurol 24, 361-4 (2009); and Ehninger, D., et al., Nat Med 14, 843-8 (2008)). Recently, this idea has been supported by a case of a TSC patient with a genetically verified mutation in TSC2, suffering from epilepsy and developmental delay, but lacking the hallmark cortical tubers (Zhang, H., et al., J Clin Invest 112, 1223-33 (2003)).

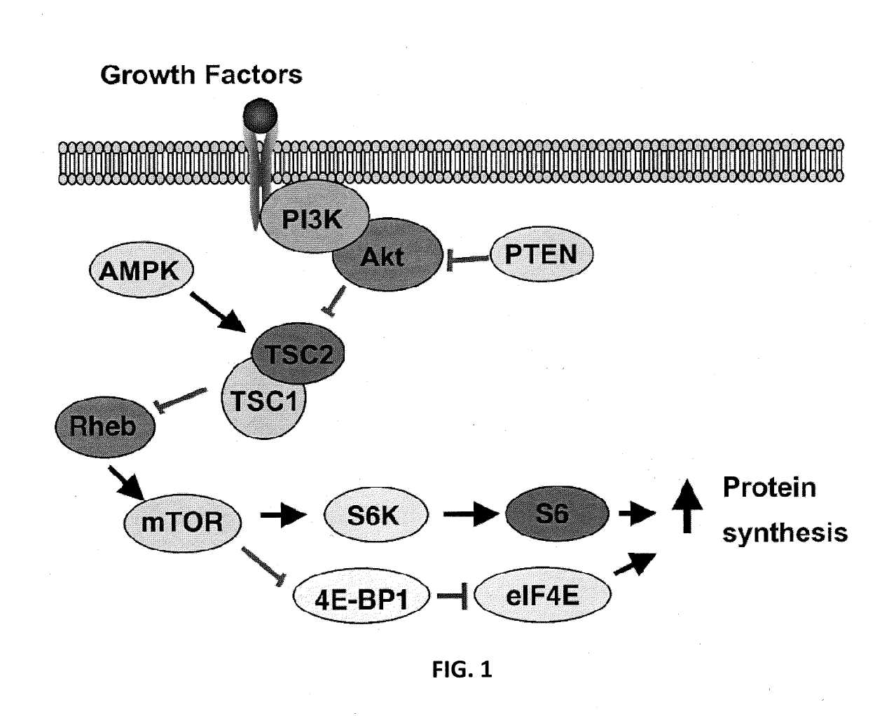

In order to understand the nature of the cognitive deficits observed in TSC and develop better treatments for the disorder, the function of the TSC1 and TSC2 gene products at the molecular/cellular level must be understood. TSC1 and TSC2 gene products form a protein complex that plays a major role in the well-studied insulin/growth factor-PI3kinase-Akt intracellular signaling pathway (Job, C., et al., Proc Natl Acad Sci USA 98, 13037-42 (2001); Todd, P. K., et al., Proc Natl Acad Sci USA 100, 14374-8 (2003); and Weiler, I. J., et al., Proc Natl Acad Sci USA 90, 7168-71 (1993)) (FIG. 1). Normally, one of the major cellular functions of the TSC1/TSC2 protein complex is to limit protein synthesis by inhibiting a Ras family GTPase, Rheb. Rheb and its downstream effector, mTOR, act as master regulators of protein synthesis and cell growth. The regulation of protein synthesis is necessary for many functions in the brain and body. The long-term maintenance of synaptic plasticity requires the synthesis of new proteins. Persistent modification of synaptic strengths is thought to be the neural basis of learning and memory, and improper regulation of synaptic protein synthesis leads to altered synaptic plasticity and adversely affects learning, memory and cognition (Gold, P. E., et al., Introduction. Neurobiol Learn Mem 89, 199-200 (2008)). Dysregulated synaptic protein synthesis may be a critical factor in the learning and cognitive deficiencies seen in TSC.

Unlike the fragile X knock out (KO) mouse, the data described herein show a deficient (decreased) Gp1 mGluR dependent plasticity and protein synthesis in Tsc2^{+/-} mice, an art-recognized animal model of TSC that presents cognitive and memory impairments similar to humans with TSC. Positive (up-regulation or activation) modulation of mGluR5 activity, as well as inhibition of mTOR activity can rescue the plasticity defect (FIG. 2), which may be indicative of a means to treat cognitive impairments in subjects with TSC by normalizing synaptic protein synthesis. Alterations in mGluR dependent plasticity and protein synthesis are causally related to the cognitive dysfunctions seen in TSC and augmenting (increasing) mGluR5 function by methods of the invention with compositions that activate Group 1 mGluR, including Group 1 mGluR agonists and positive allosteric modulators (PAMs), such as mGluR5 PAMs and/or mGluR1 PAMs, may ameliorate the synaptic and behavioral deficits seen in TSC.

In another embodiment, the subject treated by the methods of the invention that has elevated signaling of mTOR has PTEN hamartoma syndrome (PHTS). PTEN refers to “phosphatase and tensin homolog.” PHTS is a spectrum of disorders characterized by multiple hamartomas that can affect various areas of the body. Hamartoma is a general term for benign tumor-like malformations that can affect any area of the body and are composed of mature cells and tissue normally found in the affected area. PHTS includes Cowden syndrome (CS), Bannayan-Riley-Ruvalcaba syndrome (BRRS), Proteus syndrome (PS) and Proteus-like syndrome.

PHTS includes virtually all cases of Cowden syndrome (also known as multiple hamartoma syndrome) and a percentage of cases of Bannayan-Riley-Ruvalcaba syndrome, Proteus syndrome and Proteus-like syndrome (i.e., those associated with mutations of the PTEN gene).

Cowden syndrome is an under-diagnosed genetic disorder characterized by the development of multiple, benign tumor-like malformations (hamartomas) in various areas of the body. Affected individuals also have a predisposition to developing certain cancers, especially cancer of the breast, thyroid and endometrium. The specific symptoms of Cowden syndrome vary from case to case.

Bannayan-Riley-Ruvalcaba syndrome is characterized by an abnormally large head (macrocephaly), the development of multiple benign growths (hamartomatous polyps) in the intestines (intestinal polyposis), benign tumors just below the skin consisting of fatty tissue (lipomas) and excessive growth before and after birth.

Proteus syndrome is a rare, complex growth disorder characterized by disproportionate overgrowth of various parts of the body. Tissue of the bone, skin, central nervous system and eye and connective tissue are most often affected.

Proteus-like syndrome is used to describe individuals with significant features of Proteus syndrome, but who fail to meet the specific diagnostic criteria for the disorder, for Cowden syndrome and for Bannayan-Riley-Ruvalcaba syndrome.

PHTS is inherited as an autosomal dominant trait caused by mutations in the PTEN gene, an autosomal dominant tumor suppressor gene, located on chromosome 10 at position q23.3. PTEN is a protein that, in humans, is encoded by the PTEN gene. PTEN mediates cell cycle arrest and apoptosis. When both copies of the PTEN gene are altered within a cell, the affected cell may divide uncontrollably and escape programmed cell death. These abnormal cells can accumulate, leading to the formation of hamartomas that characterize PHTS.

The protein encoded by the PTEN gene is a phosphatidylinositol-3,4,5-trisphosphate 3-phosphatase. It contains a tensin like domain and a catalytic domain similar to that of the dual specificity protein tyrosine phosphatases. Unlike most of the protein tyrosine phosphatases, the protein encoded by the PTEN gene preferentially dephosphorylates phosphoinositide substrates and negatively regulates intracellular levels of phosphatidylinositol-3,4,5-trisphosphate in cells, thereby functioning as a tumor suppressor by negatively regulating Akt/PKB signaling pathway.

A preliminary diagnosis of PHTS disorders can be made based on the presence of a certain number and type of clinical features in an individual, which are known to one of skill in the art and are generally described herein. A definitive diagnosis of PHTS is made when an alteration in the PTEN gene is identified by genetic testing. Commercially available tests for genetic mutations in the PTEN gene are available, for example, from Ambry Genetics, Aliso Viejo, Calif. (THE AMBRY TEST®). The genetic test assessing mutations in the PTEN gene and its protein product.

Similar to mutations in TSC1 and/or TSC2 genes, mutations in the PTEN gene product may result in an elevation in mTOR cell signaling and, in turn, phosphorylation of FMRP by, for example, activation of S6Kinase, that suppresses synaptic protein synthesis and synaptic function, as depicted in FIG. 2. In the presence of an inhibitory molecule, such as the protein product of the PTEN, TSC1 and TSC2 genes, mTOR activity is inhibited (as shown by the “X” in FIG. 2), which, in turn, may result in the inhibition of fragile X mental retardation protein (FMRP) and, consequently, normal postsynaptic protein synthesis. In the absence of molecules that inhibit mTOR signaling, or molecules that maintain normal mTOR signaling, mTOR signaling may be elevated, FMRP is activated by phosphorylation and normal synaptic protein synthesis suppressed by activated FMRP, which may contribute to cognitive impairments and behavioral disorders associated with an elevation in mTOR signaling. Optimal protein synthesis is critical to optimizing synaptic function and, where postsynaptic protein synthesis is suppressed by elevated mTOR cell signaling, it may be normalized by compounds that activate Group I mGluR signaling, including Group I mGluR positive allosteric modulators (PAM).

In a further embodiment, subjects treated by the methods of the invention can include subjects that have a decreased LTD in postsynaptic neurons, in particular, dendrites involved in glutamate neuronal signaling. Likewise, subjects treated by the methods of the invention can include subjects that do not respond or can not be treated with Group I mGluR antagonists, Group I mGluR negative allosteric modulators (NAM) or compounds that otherwise inactivate or decrease Group I mGluR cell signaling.

In another embodiment, subjects treated by the methods of the invention can include subjects with an autism spectrum disorder that is characterized by elevated mTOR cell signaling, such as a subject with TSC that has autism.

Autism spectrum disorder is a developmental disorder that affects an individual's ability to communicate, form relationships with others and respond appropriately to the environment. Some individuals with autism spectrum disorder are high functioning, with speech and intelligence within normal range. Other individuals with autism spectrum disorder may be nonverbal and/or have varying degrees of mental retardation. Autism spectrum disorder can include idiopathic autism (e.g., autism of unknown origin). One of skill in the art would be able to diagnosis an individual with autism spectrum disorder, employing well-known clinical criteria as described, for example, in Diagnostic and Statistical Manual of Mental Disorders (DSMMD) (4th ed., pp. 70-71) Washington, D.C., American Psychiatric, 1994.

In an embodiment, the compound employed in the methods of the invention that activates a Group 1 mGluR includes a Group 1 mGluR agonist. In another embodiment, the compound employed in the methods of the invention that activates a Group 1 mGluR includes a Group 1 mGluR positive allosteric modulator (PAM).

Metabotrophic glutamate receptors (mGluRs) are a heterogeneous family of glutamate G-protein coupled receptors that can regulate local synaptic protein synthesis (Job, C., et al., Proc Natl Acad Sci USA 98, 13037-42 (2001); Todd, P. K., et al., Proc Natl Acad Sci USA 100, 14374-8 (2003); and Weiler, I. J., et al., Proc Natl Acad Sci USA 90, 7168-71 (1993)) mGluRs are classified into three groups. Group 1 (Gp1) receptors (mGluR1 and mGluR5) can be coupled to stimulation of phospholipase C resulting in phosphoinositide hydrolysis and elevation of intracellular calcium levels, modulation of ion channels (e.g., potassium channels, calcium channels, non-selective cation channels) and N-methyl-D-aspartate (NMDA) receptors. mGluR5 can be present on a postsynaptic neuron. mGluR1 can be present on a presynaptic neuron and/or a postsynaptic neuron. Group 2 receptors (mGluR2 and mGluR3) and Group 3 receptors (mGluRs 4, 6, 7, and 8) inhibit cAMP formation and G-protein-activated inward rectifying potassium channels. Group 2 mGluRs and Group 3 mGluRs are negatively coupled to adenylyl cyclase, generally present on presynaptic neurons, but can be present on postsynaptic neurons and function as presynaptic autoreceptors to reduce glutamate release from presynaptic neurons. Glutamate is the major excitatory neurotransmitter in the brain and glutamate receptors are widely expressed in the brain.

mGluR-dependent translation can play a role in forms of synaptic plasticity, including a type of long term depression (LTD) in the hippocampus, an area of the brain known to be important for learning and memory. This form of hippocampal LTD is dependent on mGluR5 and requires rapid protein synthesis (Huber, K. M., et al., Proc Natl Acad Sci USA 99, 7746-50 (2002); Huber, K. M., et al., Science 288, 1254-7 (2000)) mGluR LTD is increased in the mouse model of fragile X syndrome, which results from a genetic mutation and in which mental retardation and autism are present.

Compositions employed in the methods of the invention to treat conditions having an elevation in mTOR signaling, such as TSC, activate mGluR signaling. “Activate,” in reference to mGluR signaling as used herein, means that the compositions prompt, promote or augment cell signaling through metabotrophic glutamate receptors. Compositions that activate mGluR include, for example, at least one member selected from the group consisting of an mGluR agonist and an mGluR positive allosteric modulator.

A mGluR agonist (e.g., Group 1 mGluR agonist, mGluR1 agonist, mGluR5 agonist) mimics the effect of a ligand (e.g., glutamate) to thereby activate mGluR1 and/or mGluR5. The mGluR agonist may act at the level of ligand-receptor interaction, such as by competitively or non-competitively (e.g., allosterically) activating ligand binding. The mGluR agonist (e.g., mGluR1 agonist, mGluR5 agonist) can be, for example, a chemical agonist or a pharmacokinetic agonist. The mGluR agonist may act downstream of the receptor, such as by activating receptor interaction with a G-protein or subsequent cell signaling events associated with G-protein activation, such as activation of PLC, an increase in intracellular calcium, the production of or levels of cAMP or adenyl cyclase and stimulation or modulation of ion channels (e.g., potassium channels, calcium channels).

Exemplary mGluR agonists for use in the invention can include at least one compound of Formulas I-III.

Quisqualic acid/L-qusiqualate (Formula I) (Tocris, Sigma) ((L)-(+)-a-Amino-3,5-dioxo-1,2,4-oxadiazoline-2-propanic acid), which is a Group 1 selective agonist (mGluR1, mGluR5) (Brauner-Osborne, H., et al., Br J Pharmacol, 123(2): p. 269-74 (1998); Watkins, J. C., et al., Trends Pharmacol Sci, 11(1): p. 25-33 (1990); Watkins, J. C., et al., Adv Exp Med Biol, 268: p. 49-55 (1990)).

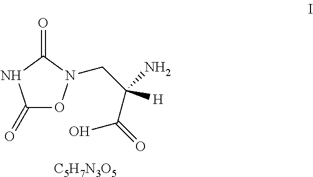

(S)-3,5-DHPG (Formula II) (Tocris, Sigma) ((S)-3,5-Dihydroxyphenylglycine), which is a Group 1 selective agonist (mGluR1, mGluR5) (Schoepp, D. D., et al., J Neurochem., 63(2): p. 769-72 (1994); Contractor, A., et al., Proc Natl Acad Sci USA, 95(15): p. 8969-74 (1998); Wisniewski, K., et al., CNS Drug Rev, 8(1): p. 101-16 (2002)).

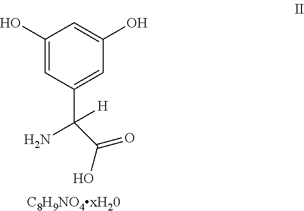

CHPG (Formula III) (Tocris, Sigma) ((RS)-2-chloro-5-hydroxyphenylglycine), which is a mGluR5 agonist (Doherty, A. J., et al., Neuropharmacology, 36(2): p. 265-7 (1997)).

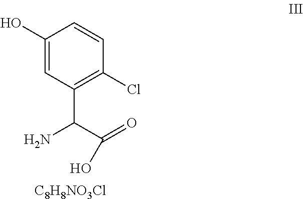

A positive allosteric modulator (PAM) of mGluR, in particular a Group 1 mGluR PAM, indirectly activates mGluR by enhancing sensitivity of the mGluR to ligands (e.g., glutamate) by binding to allosteric sites in the seven-transmembrane-spanning domains of mGluR.

Exemplary mGluR5PAMs for use in the methods of the invention can include at least one compound listed below (Formulas IV-XII):

DFB (Formula IV) (Sigma, Tocris) ([(3-Fluorophenyl)methylene]hydrazone-3-fluorobenzaldeyhde), which is a mGluR5 PAM (O'Brien, J. A., et al., Mol Pharmacol, 64(3): p. 731-40 (2003)).

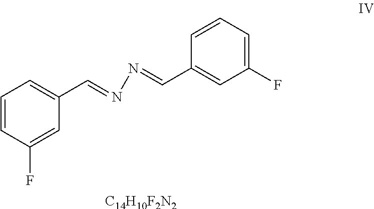

CPPHA (Formula V) (Sigma) (N-{4-chloro-2-[(1,3-dioxo-1,3-dihydro-2H-isoindol-2-yl) methyl]phenyl}-2-hydroxy/benzamide), which is a mGluR5 PAM (O'Brien, J. A., et al., J Pharmacol Exp Ther, 309(2): p. 568-77 (2004)).

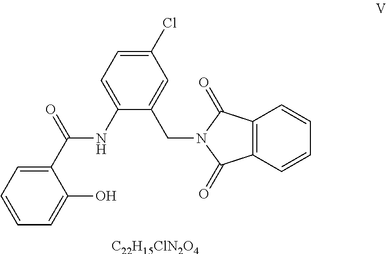

CDPPB (Formula VI) (Tocris, Calbiochem) (3-cyano-N-(1,3-diphenyl-1H-pyrazol-5-yl)benzamide), which is a mGluR5 PAM (Ayala, J. E., et al., Neuropsychopharmacology, 34(9): p. 2057-71 (2009); Uslaner, J. M., et al., Neuropharmacology, 57(5-6): p. 531-8 (2009); Kinney, G. G., et al., J Pharmacol Exp Ther, 313(1): p. 199-206 (2005); Lindsley, C. W., et al., J Med Chem, 47(24): p. 5825-8 (2004)).

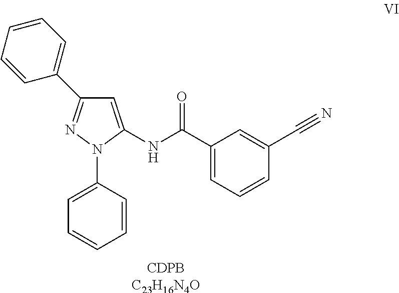

VU-29 (Formula VII) (4-nitro-N-(1,3-diphenyl-1H-pyrazol-5-yl)benzamide), which is a mGluR5 PAM (Ayala, J. E., et al., Neuropsychopharmacology, 34(9): p. 2057-71 (2009); Chen, Y., et al., Mol Pharmacol, 71(5): p. 1389-98 (2007); de Paulis, T., et al., J Med Chem, 49(11): p. 3332-44 (2006)).

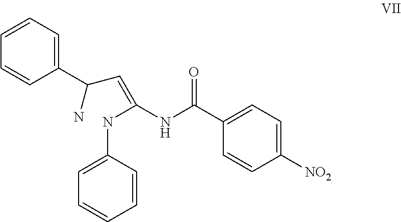

ADX47273 (Formula VIII) ([S-(4-fluoro-phenyl)-{3-[3-(4-fluoro-phenyl)-[1,2,45]oxadiazol-5-yl]-piperidin-1-yl}-methanone]), which is a mGluR5 PAM (Liu, F., et al., J Pharmacol Exp Ther, 327(3): p. 827-39 (2008)).

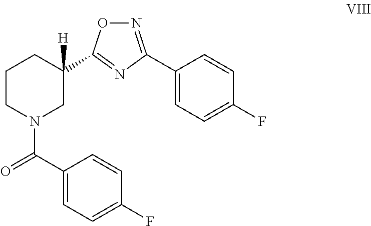

Exemplary mGluR1 PAMs for use in the methods of the invention can include at least one compound listed below (Formulas IX-XII):

Ro 67-7476 (Formula IX) ((S)-2-(4-fluorophenyl)-1-(toluene-4-sulfonyl)pyridine), which is a mGluR1 PAM (Wisniewski, K., et al., CNS Drug Rev, 8(1): p. 101-16 (2002); Doherty, A. J., et al., Neuropharmacology, 36(2): p. 265-7 (1997); Knoflach, F., et al., Proc Natl Acad Sci USA, 98(23): p. 13402-7 (2001)).

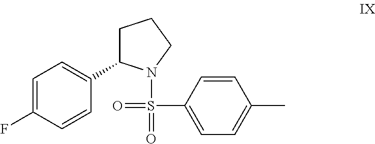

Ro 67-4853 (Formula X) (Butyl (9H-xanthene-9-carbonyl)carbamate), which is a mGluR1 PAM (Wichmann, J., et al., Farmaco, 57(12): p. 989-92 (2002)).

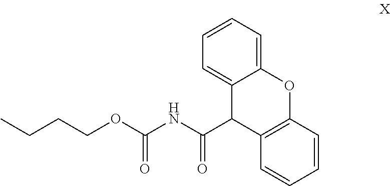

Ro 01-6128 (Formula XI) (Diphenylacetyl-carbamic acid ethyl ester), which is a mGluR1 PAM (Wichmann, J., et al., Farmaco, 57(12): p. 989-92 (2002)).

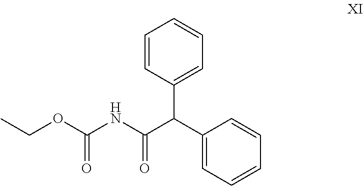

VU-71 (Formula XII) (4-nitro-N-(1,4-diphenyl-1H-pyrzol-5-yl)benzamide), which is a mGluR1 PAM (Hemstapat, K., et al., Mol Pharmacol, 70(2): p. 616-26 (2006)).

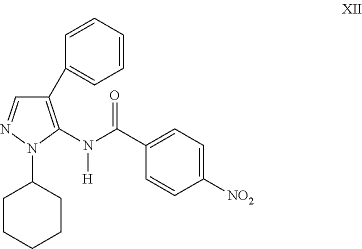

In an embodiment, the subjects that have an elevation in mTOR signaling, such as subjects with TSC, treated by the methods of the invention also have autism.

Subjects treated by the methods of the invention include humans (also referred to as “patients”). The humans treated by the methods of the invention can be children. Children can be treated at any age of life, including infancy and adolescence. Humans treated by the methods of the invention can be adults (greater than 18 years of age) and elderly humans (greater than 65 years of age).

Subjects treated by the methods of the invention can have a cognitive impairment, such as an impairment in attention, executive function, reaction time, learning, information processing, conceptualization, problem solving, verbal fluency or memory (e.g., memory consolidation, short-term memory, working memory, long-term memory, declarative memory or procedural memory).

Impairment in a cognitive function treated by the methods described herein can be an impairment in attention, which is the capacity or process of selecting out of the totality of available sensory or affective stimuli, those stimuli that are most appropriate or desirable for focus at a given time (Kinchla, R. A., et al., Annu. Rev. Psychol. 43:711-742 (1992)). The impairment in a cognitive process can be an impairment in executive function, which are neuropsychological functions such as decision making, planning, initiative, assigning priority, sequencing, motor control, emotional regulation, inhibition, problem solving, planning, impulse control, establishing goals, monitoring results of action and self-correcting (Elliott, R., Br. Med. Bull. 65:49-59 (2003)). The cognitive impairment can be an impairment in alertness, wakefulness, arousal, vigilance, and reaction time information processing, conceptualization, problem solving and/or verbal fluency. One of skill in the art would be capable of identifying and evaluating the impairment in a cognitive function in the individual employing well-known tests, such as Rey Auditory and Verbal Learning Test (RAVLT); a Children's Memory Scale (CMS); a Contextual Memory Test; a Continuous Recognition Memory Test (CMRT); First-Last Name Association (Youngjohn J. R., et al., Archives of Clinical Neuropsychology 6:287-300 (1991)); Wechsler Memory Scale-Revised (Wechsler, D., Wechsler Memory Scale-Revised Manual, NY, N.Y., The Psychological Corp. (1987)); Cognitive Drug Research (CDR) Computerized Assessment Battery-Wesnes; Buschke's Selective Reminder Test (Buschke, H., et al., Neurology 24:1019-1025 (1974)); Telephone Dialing Test; and Brief Visuospatial Memory Test-Revised.

In an embodiment, the subjects treated by the methods of the invention can have a seizure disorder, such as at least one seizure disorder selected from the group consisting of an audiogenic seizure and an epileptic seizure.

A seizure disorder can be caused by abnormal electrical conduction in the brain, resulting in the abrupt onset of transient neurologic symptoms such as involuntary muscle movements, sensory disturbances and altered consciousness. Seizure disorders can be categorized based on whether the seizure is localized in a particular region of the brain (partial or focal onset seizures) or distributed throughout the brain (generalized seizures). Partial seizures are further divided on the extent to which consciousness is affected (simple partial seizures and complex partial seizures). If consciousness is unaffected, then it is a simple partial seizure; otherwise it is a complex partial seizure. A partial seizure may spread within the brain, which is referred to as secondary generalization. Generalized seizures are divided according to the effect on the body, but all involve loss of consciousness. These include absence, myoclonic, clonic, tonic, tonic—clonic and atonic seizures. A mixed seizure is defined as the existence of both generalized and partial seizures in the same patient. An audiogenic seizure can be brought on by sound, for example, abrupt noise or loud noise. An epileptic seizure occurs in epilepsy, a common chronic neurological disorder characterized by recurrent unprovoked seizures.

The compositions that activate mGluR employed in the methods of the invention can be administered in a dose of between about 0.1 mg/kg to about 1 mg/kg body weight; between about 1 mg/kg to about 5 mg/kg body weight; or between about 5 mg/kg to about 15 mg/kg body weight. The compositions can be administered in doses of about 0.01 mg, about 0.05 mg, about 0.1 mg, about 0.5 mg, about 1 mg, about 2 mg, about 10 mg, about 25 mg, about 50 mg, about 100 mg, about 200 mg, about 250 mg, about 300 mg, about 400 mg, about 500 mg, about 600 mg, about 700 mg, about 900 mg, about 1000 mg, about 1200 mg, about 1400 mg, about 1600 mg, about 2000 mg, about 500 mg, about 10,000 mg, about 50,000 mg or about 100,000 mg. The compositions can be administered once a day or multiple (e.g., two, three, four, five) times per day.

The compounds employed in the methods of the invention can be administered to a subject with (e.g., before, concomitantly, sequentially or after) administration of other compounds that are employed to treat a particular disorder or condition in the subject. For example, the compositions of the invention can be administered with at least one member selected from the group consisting of an anti-anxiety treatment and an anti-seizure treatment.

The compositions employed in the methods of the invention can be administered to the subject acutely (briefly or short-term) or chronically (prolonged or long-term).

The subject having an elevation in mTOR signaling treated by the methods of the invention can also have a lower than average intelligence or mental retardation. Intelligence describes the subject's ability to think, learn and solve problems. A subject having an elevation in mTOR signaling with mental retardation may have difficulty learning, may take longer to learn social skills, such as how to communicate, and may be less able to care for himself or herself and to live on his or her own as an adult.

The compositions employed in the methods of the invention can be administered to subjects, in particular humans, by multiple routes of administration (e.g., intramuscular, oral, intranasal, inhalation, topical, transdermal). The compositions (e.g., mGluR agonist, mGluR PAM) employed in the methods of the invention can be administered alone or as admixtures with conventional excipients, for example, pharmaceutically, or physiologically, acceptable organic, or inorganic carrier substances suitable for enteral or parenteral application which do not deleteriously react with the compounds) administered to the subject. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds employed in the methods of the invention. The preparations can also be combined, when desired, with other active substances to reduce metabolic degradation. The compositions employed in the methods of the invention, alone, or when combined with an admixture, can be administered in a single or in more than one dose (multiple doses) over a period of time to confer the desired effect (e.g., improve cognition).

When parenteral application is needed or desired, particularly suitable admixtures for the compounds employed in the methods of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds for use in the methods of the invention can also be incorporated into liposomes or administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309.

The dosage and frequency (single or multiple doses) administered to the subject can vary depending upon a variety of factors, including the severity of a condition, such as severity of cognitive impairment, mental retardation, autism and seizure disorder; the route of administration of the composition; age, gender, health and body weight, types of concurrent treatment (e.g., behavioral modification, anticonvulsants), complications from, for example, a seizure disorder, impaired cognitive function; or other health-related problems. Other therapeutic regimens or agents can be used in conjunction with the methods of the present invention. For example, the administration of the compositions employed in the methods of the invention can be accompanied by behavioral modifications and anti-seizure medications. Adjustment and manipulation of established dosages (e.g., frequency and duration) are well within the ability of those skilled in the art.

An “effective amount,” also referred to herein as a “therapeutically effective amount,” when referring to the amount of a composition that activates Group 1 mGluR signaling, such as a mGluR agonist or mGluR PAM, means that amount, or dose, of a compound, composition, mGluR agonist or mGluR PAM that, when administered to a subject, is sufficient for therapeutic efficacy (e.g., an amount sufficient decrease to exhibit a clinical improvement in a behavior or cognitive score; alleviate a seizure disorder).

Experimental assessment of compounds employed in methods of the invention can be made using preclinical techniques, such as comparing wild type and Tsc2^{+/-} mice, as described herein. For example, contextual fear conditioning, the Morris Water Maze Task, Ocular Dominance Plasticity evaluation and the Five Choice Serial Reaction Time Task, can be employed.

Contextual fear conditioning is a common indicator of hippocampal dependent learning. A recent study has shown that Tsc2^{+/-} mice trained in the context fear-conditioning task have a deficit in the ability to discriminate between the training context and a novel context (Ehninger, D., et al., Nat Med 14, 843-8 (2008)).

The Morris water maze task is another well-established measure of hippocampal-dependent learning. Subjects with an elevation in mTOR signaling may have impaired performance in this task. As with the contextual fear-conditioning task, this paradigm provides another measure of the relationship between the electrophysiological and behavioral impairments in subjects with an elevation in mTOR signaling, and the relationship between mTOR and mGluR signaling in these impairments. The ability of mGluR5 PAM treatment to enhance the performance of mice with an elevation in mTOR signaling in the Morris water maze task can be determined as previously described (Ehninger, D., et al., Nat Med 14, 843-8 (2008)).

Briefly, wildtype and mice with an elevation in mTOR signaling (8-12 week old) can be trained on the hidden platform version of the Morris water maze with four training trials occurring per day for 5 consecutive days. The escape platform is hidden 1 cm under the water surface in a constant location. Mice are released into the pool from one of several locations. Training trials end when either the mouse reaches the platform or about 60 sec has elapsed. Mice will remain on the platform for about 15 sec before being removed from the pool. After training is completed a probe trial can be given, during which the platform is removed. Spatial learning is assessed by quadrant occupancy and target crossing during the probe trial.

Injections of compositions that activate Group 1 mGluR signaling can be given daily, 30 minutes prior to each training session. No injections can be given on the day of the probe trial. The four groups (wildtype+vehicle, elevated mTOR+vehicle, wildtype+drug, and elevated mTOR+drug) can be assessed. All experiments are performed blind to genotype and include yoked controls for genotype and treatment.

The visual cortex is a well-established model for experience-dependent development of cortical circuitry and function. In recent years, mice have become the preferred species for these studies, and the mechanistic understanding of visual cortical plasticity has advanced considerably. Brief monocular deprivation (MD) causes first depression of excitatory transmission of synapses dedicated to the deprived eye, followed by a compensatory potentiation of synapses dedicated to the non-deprived eye (Frenkel, M. Y., et al., Neuron 44, 917-23 (2004)). The normal cortical response to brief monocular deprivation (MD) engages mechanisms of LTD, long-term potentiation (LTP), metaplasticity, synaptic homeostasis, as well as inhibitory and structural plasticity (Smith, G. B., et al., Philos Trans R Soc Lond B Biol Sci 364, 357-67 (2009)). In addition, visual cortical responses can be enhanced following selective visual experience, a phenomenon that likely reveals mechanisms of perceptual learning (Frenkel, M. Y., et al., Neuron 51, 339-49 (2006)). Therapeutically relevant insights have already been gained by using the mouse visual cortex model in genetically defined developmental brain disorders, such as fragile X (Dolen, G., et al., Neuron 56, 955-62 (2007)) and Angelman (Yashiro, K., et al., Nat Neurosci 12, 777-83 (2009)) syndromes. Such assays may characterize any deficits mice with elevation in mTOR signaling may have in cortical synaptic plasticity and assess the effect of treatment with activators of Group 1 mGluRs, such as Group 1 mGluR PAM on such impairments.

The sophisticated understanding of visual cortical plasticity makes this a suitable model to discover synaptic pathophysiology in genetic disorders that can be modeled in mice. OD shift after MD appeared to be accelerated, suggesting “hyperplasticity”, which, remarkably, could be corrected simply by reducing mTOR signaling.

Visually evoked potentials (VEPs) are made following 1, 3, and 7 days of MD. Brief MD causes selective depression of deprived eye responses, clearly apparent at 1 day and reaching an asymptote at 3 days. Thus, these deprivation protocols measure the rate and amount of synaptic depression. Seven (7) days of MD causes a compensatory increase in responses from the non-deprived eye. VEPs are a sensitive measure of visual performance, and can be used to measure baseline visual acuity and contrast sensitivity. This approach can be employed with other recording methods, including single unit recordings and optical imaging to assess the gross organization of visual cortex and receptive field properties in the mutant mice. For VEP recordings, tungsten microelectrodes are implanted in the binocular visual cortex about 450 μm below the cortical surface while animals are anesthetized with 50 mg/kg ketamine and 10 mg/kg xylazine i.p., and reference electrodes are placed bilaterally in prefrontal cortex. Sutures are used for monocular deprivation. Following isofluorane anesthesia, eyelids are trimmed and three stitches are placed, closing the entire lid.

Mice are monitored daily to ensure that the sutured eye remained completely shut and uninfected. Following the three day deprivation, stitches are removed and the eye is flushed with saline. Stimuli consist of full-field sine-wave gratings of 0% and 100% contrast, square reversing at 1 Hz, and presented at 0.05 cycles/degree on a computer monitor. VEPs are elicited by either horizontal or vertical bars. The display is positioned 20 cm in front of the mouse and centered on the midline, thereby occupying 92°×66° of the visual field. VEP recordings are conducted in awake head-fixed mice. The animals are alert and still during recording. Visual stimuli are presented to left and right eyes randomly. A total of about 100 to about 200 stimuli are presented per each condition. VEP amplitude is quantified by measuring peak-to-peak response amplitude, and data are normalized to the day 0 ipsilateral eye value. Statistical analysis will be performed using MANOVA® followed by post-hoc analysis if a main effect of genotype is observed.

In the awake mouse, daily presentation of contrast-reversing sinusoidal grating stimuli at a single orientation leads to a specific potentiation of the VEPs elicited by those stimuli, a phenomenon termed SRP (for “stimulus-selective response potentiation”). SRP shares similar properties to those described for some forms of human perceptual learning (Karni, A., et al., Curr Opin Neurobiol 7, 530-5 (1997)). Mechanistically, this naturally-occurring enhancement of synaptic strength shares several properties with long-term potentiation (LTP) as it is: input specific, NMDA receptor dependent, rapidly induced, saturable, long-lasting, and protein synthesis dependent (Frenkel, M. Y., et al., Neuron 51, 339-49 (2006)).

Established techniques can assess the ability of mice with an elevation in mTOR signaling to exhibit perceptual learning and can determine if there are any impairments in a naturally occurring form of cortical synaptic strengthening. Upon identifying any deficits in SRP, such deficients can be by the methods described herein. The major advantage of SRP for these pharmacological rescue experiments is that the synaptic modifications are induced by brief episodes of visual experience, lasting no more than one hour. Thus, unlike ocular dominance plasticity, which would require chronic treatments, SRP can be manipulated with acute injections of test compounds. SRP is that an analagous phenomenon has been observed in humans (Ross, R. M., et al., Brain Res Bull 76, 97-101 (2008)).

Chronic implantation of visual cortical recording electrodes and habituation to the recording apparatus can be performed as described above. Animals are subjected to daily exposure to sinusoidal grating stimuli (about 0.05 cyc/deg, 100% contrast) of a fixed orientation alternating in phase with a fixed temporal frequency of presentation of 1 Hz. During each daily training session visual stimuli (about 400 stimuli) will be presented randomly binocularly and to the left and right eyes. Presentation of visual stimuli will be performed daily until SRP is saturated, which in wild type animals, has been shown to occur within 4-5 days (Frenkel, M. Y., et al., Neuron 51, 339-49 (2006)) VEP amplitude is quantified by measuring trough to peak response amplitude, as described previously. Responses to stimuli of 0% contrast are also collected to measure activity not evoked by patterned visual stimulation.

When differences in SRP in mice with an elevation in mTOR signaling as compared to wildtype controls are present, subjects can receive i.p. compositions employed in the methods of the invention or the appropriate vehicle. Injections are given about 30 to about 120 minutes before the selective visual experience, and response enhancement will be assessed about 24 hours later. Experimental groups (wildtype+vehicle, elevated mTOR+vehicle, wildtype+drug, and elevated mTOR+drug) are assessed as described herein. All experiments will be performed blind to genotype and include yoked controls for genotype and treatment. Cognitive impairments associated with an elevation in mTOR signaling in TSC include deficits in executive-attentional function (Prather, P., et al., J Child Neurol 19, 666-74 (2004); and Gillberg, I. C., et al., Dev Med Child Neurol 36, 50-6 (1994)). Executive function refers to a set of cognitive skills involved in attention, inhibitory control and cognitive flexibility. Despite extensive clinical research into this core deficit in TSC and other conditions characterized by an elevation in mTOR signaling, impairments in executive dysfunction have received little attention in pre-clinical research. A battery of behavioral measures to assess executive function in subjects with an elevation in mTOR signaling and to subsequently utilize this battery to investigate the role of metabotropic glutamate receptor signaling in this core deficit can be performed.

The five choice serial reaction time task (5CSRTT) is a well established operant conditioning paradigm that is used to assess executive function in rodents (Ehninger, D., et al., Nat Med 14, 843-8 (2008)). It was developed as a rodent equivalent to the human continuous performance test (Chudasama, Y., et al., Biol Psychol 73, 19-38 (2006); and Wrenn, C. C., et al., Pharmacol Biochem Behav 83, 428-40 (2006)). The principle purpose of the 5CSRTT is to assess sustained attention, but it can also be modified to measure impulsivity and repetitive responding.

In this task, mice are placed in an operant conditioning chamber (Med Associates, USA) equipped with a food magazine and pellet dispenser, stimulus light, house light and five nosepoke apertures. Each of these nosepoke apertures can be individually illuminated to provide a brief visual stimulus, and mice are required to continuously monitor the nosepoke apertures for these visual stimuli in order to complete the task. Mice are habituated to the apparatus and trained to nosepoke in an illuminated aperture to obtain a food reward. At the beginning of each trial, one of the five apertures are randomly designated as the active aperture and illuminated for that trial. The mouse are required to make a response in the illuminated aperture (correct response) to receive a food pellet. Responses in the non-illuminated apertures (incorrect responses), failure to respond during or within 5 sec after termination of the stimulus (omission errors), and responses in one of the apertures prior to onset of the stimulus (anticipatory errors) results in a 2 sec timeout, during which the house light and all other lights are turned off.

Even if mice are able to learn the 5CSRTT, several additional aspects of the animals' performance can still be assessed. Anticipatory responses (defined by premature nose pokes) are thought to be analogous to impulsivity in humans. Perseverative responses (additional nose pokes following a correct response before a new trial is initiated) are thought to be analogous to compulsive behaviors in human. In this task, the reward rule can be change to aperture location rather than stimulus location to assess reversal learning. While the 5CSRTT is a complicated task it is also very flexible, allowing for many cognitive features to be examined.

Treatment with compositions that activate Group 1 mGluR can restore 5CSRTT performance in mice with an elevation in mTOR signaling, by normalizing mTOR signaling. There are several phases of training in the 5CSRTT and the proper timing for treatment can be determined. If mice with an elevation in mTOR signaling are impaired on the acquisition of this task, they will be given injections starting on the first day training However, if mice with an elevation in mTOR signaling can learn the 5CSRTT, but have impairment on a certain aspect of the task, then it can be initially determined if treatment given after training can acutely rescue these deficits. If not, then prolonged treatment throughout the training process will be performed.

### EXEMPLIFICATION

### Example 1

### Alterations in Neuronal Signaling

Human TSC is characterized by growth of hamartomas in multiple organs, including the brain. TSC1 or TSC2 mutations disrupt a protein complex that, among other consequences, acts to inhibit Rheb, a Ras family GTPase with high specificity for mTOR within a protein complex called mTORC1. Rheb activation of mTORC1 can stimulate mRNA translation and cell growth, and excessive activation is believed to be pathogenic in TSC (Ehninger, D., et al., Nat Med 14, 843-8 (2008)). Although some manifestations of TSC (e.g., seizures) are thought to be related to tuber growth in the cerebral cortex, others including cognitive impairment and autism have been proposed to result from abnormal signaling at synapses (deVries, P. J., et al., Trends Mol Med 13: 319 (2007)). Mice engineered to carry heterozygous loss-of-function mutations in Tsc1 or Tsc2 have been shown to have hippocampus-dependent learning and memory deficits without having tumors or seizures (Goorden, S. M., et al., Ann Neurol 62, 648-55 (2007); Ehninger, D., et al., Nat Med 14, 843-8 (2008)). Postnatal treatment of the Tsc2^{+/-} mice with the mTORC1 inhibitor rapamycin was shown to ameliorate hippocampal memory impairments suggesting the exciting possibility that some aspects of TSC might be amenable to drug therapy.

Synaptic function in the Tsc2^{+/-} mice may be related to altered synaptic protein synthesis in response to elevated mTORC1 activity. LTD is a sensitive functional read-out of synaptic mRNA translation (Huber, K. M., et al., Science 288, 1254-7 (2000)). Alterations in mGluR-dependent LTD in the hippocampus of male Tsc2^{+/-} mice are described.

### Animals

Tsc2^{+/-} male and female mutant mice on the C57B1/6J clonal background were bred with C57B1/6J WT partners to produce the WT and Tsc2^{+/-} offspring used. All experimental animals were age-matched male littermates, and were studied with the experimenter blind to genotype and treatment condition. Animals were group housed and maintained on a 12:12 hr light: dark cycle.

### Electrophysiology

Acute hippocampal slices were prepared from P25-30 animals in ice-cold dissection buffer containing (in mM): NaCl 87, Sucrose 75, KCl 2.5, NaH₂PO₄ 1.25, NaHCO₃ 25, CaCl₂ 0.5, MgSO₄ 7, Ascorbic acid 1.3, and D-glucose 10 (saturated with 95% O₂/5% CO₂). Immediately following slicing the CA3 region was removed. Slices were recovered in artificial cerebrospinal fluid (ACSF) containing (in mM): NaCl 124, KCl 5, NaH₂PO₄ 1.23, NaHCO₃ 26, CaCl₂ 2, MgCl₂ 1 and D-glucose 10 (saturated with 95% O₂/5% CO₂) at 32.5° C. for about >3 hours prior to recording.

Field recordings were performed in a submersion chamber, perfused with ACSF (about 2-3 ml/min) at 30° C. Field EPSPs (fEPSPs) were recorded in CA1 stratum radiatum with extracellular electrodes filled with ACSF. Baseline responses were evoked by stimulation of the Schaffer collaterals at 0.033 Hz with a 2-contact cluster electrode (FHC) using about a 0.2 ms stimulus yielding about 40-60% of the maximal response. fEPSP recordings were filtered at about 0.1 Hz-1 kHz, digitized at 10 kHz, and analyzed using pClamp9 (Axon Instruments). The initial slope of the response was used to assess changes in synaptic strength. Data were normalized to the baseline response and are presented as group means±SEM. LTD was measured by comparing the average response about 55-60 minutes post DHPG application to the average of the last 5 minutes of baseline.

Input output function was examined by stimulating slices with incrementally increasing current (about 20, about 40, about 80, about 120, about 200, about 300 μA) and recording the fEPSP response. Paired pulse facilitation was induced by applying two pulses at different interstimulus intervals (about 10, about 20, about 50, about 100, about 200, about 300, about 500 ms). Facilitation was measured by the ratio of the fEPSP slope of stimulus 2 to stimulus 1. NMDAR-dependent LTD was induced by delivering 900 test pulses at 1 Hz. mGluR-LTD was induced by applying R, S-Dihydroxyphenylglycine (R,S-DHPG, 50 μM) or S-Dihydroxyphenylglycine (S-DHPG, 25 μM) for about 5 minutes, the effects of which were followed for 60 minutes following treatment. In some experiments slices were incubated with the protein synthesis inhibitor cycloheximide (60 μM) for 30 minutes as follows: about 20 minutes during baseline recording, about 5 minutes during DHPG application and about 5 minutes post DHPG application.

For mGluR PAM experiments, slices were pretreated with CDPPB (10 μM) or DMSO control for about 30 minutes in same manner as above, either in the presence of cycloheximide or control ACSF. For rapamycin experiments, slices were pretreated with rapamycin (20 nM) or DMSO control, with or without cycloheximide, for at least 30 minutes prior to DHPG application and throughout the entire experiment.

Significance was determined by two-way ANOVA and post-hoc Student's t-tests. All experiments were performed blind to genotype and include interleaved controls for genotype and treatment.

### Reagents

(R,S)-3,5-dihydroxyphenylglycine (R,S-DHPG) was purchased from Tocris Biosciences (Ellisville, Mo.) and (S)-3,5-dihydroxyphenylglycine (S-DHPG) was purchased from Sigma (St. Louis, Mo.). Fresh bottles of DHPG were prepared as a 100× stock in H₂O, divided into aliquots, and stored at −80° C. Fresh stocks were made once a week. Rapamycin (EMD Biosciences, San Diego, Calif.) was prepared at 10 mM stock in DMSO and stored at −80° C. Final concentration of rapamycin was 20 nM in <0.01% DMSO. Cycloheximide (Sigma) was prepared daily at 100× stock in H₂O. For slice experiments, 3-Cyano-N-(1,3-diphenyl-1H-pyrazol-5-yl)benzamide (CDPPB, EMD Biosciences) was prepared daily at 75 mM stock in DMSO and diluted in ACSF to achieve final concentration of 10 μM in <0.1% DMSO. For in vivo experiments, CDDPB was suspended in a vehicle consisting of 20% (2-Hydroxypropyl)-β-cyclodextrin in saline. All other reagents were purchased from Sigma.

mGluR-Dependent LTD is Altered in Tsc2^{+/-} Mice

Activation of Group 1 mGluRs with the selective agonist DHPG ((R,S)-3,5-dihydroxyphenylglycine) induces LTD in area CA1 of the hippocampus by two independent mechanisms: reduced probability of presynaptic glutamate release (Fitzjohn, S. M., et al., J. Physiol. 537: 421 (2001); and Nosyreva, E. D., et al., J. Neuroscience 25: 2992 (2005)) and reduced expression of postsynaptic AMPA receptors (Nosyreva, E. D., et al., J. Neuroscience 25: 2992 (2005)). In wild type (WT) animals, the postsynaptic modification is known to require immediate translation of mRNAs available in the dendrites of hippocampal pyramidal neurons (Huber, K. M., et al., Science 288, 1254-7 (2000); and Snyder, E. M., et al., Nat. Neurosci 4: 1079 (2001)).

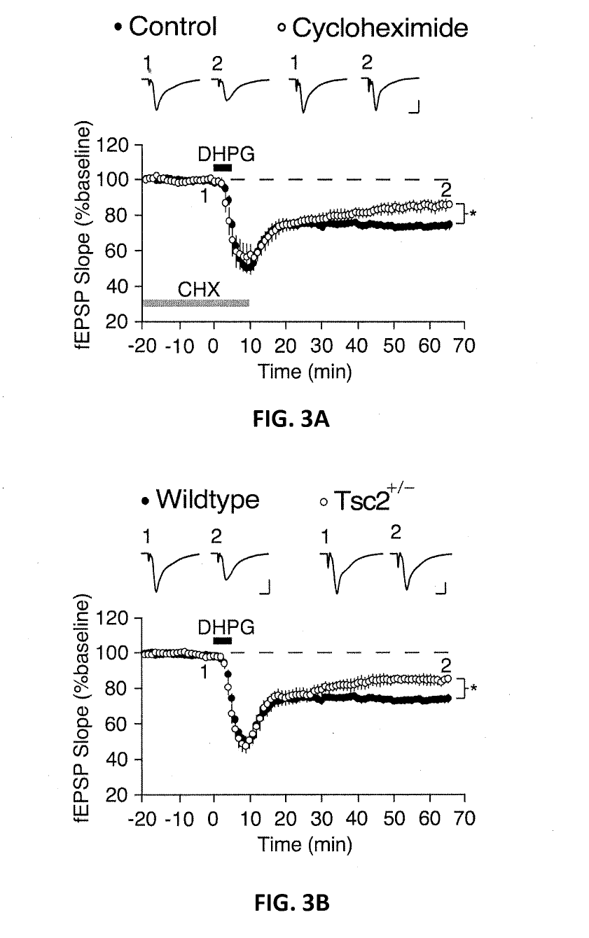

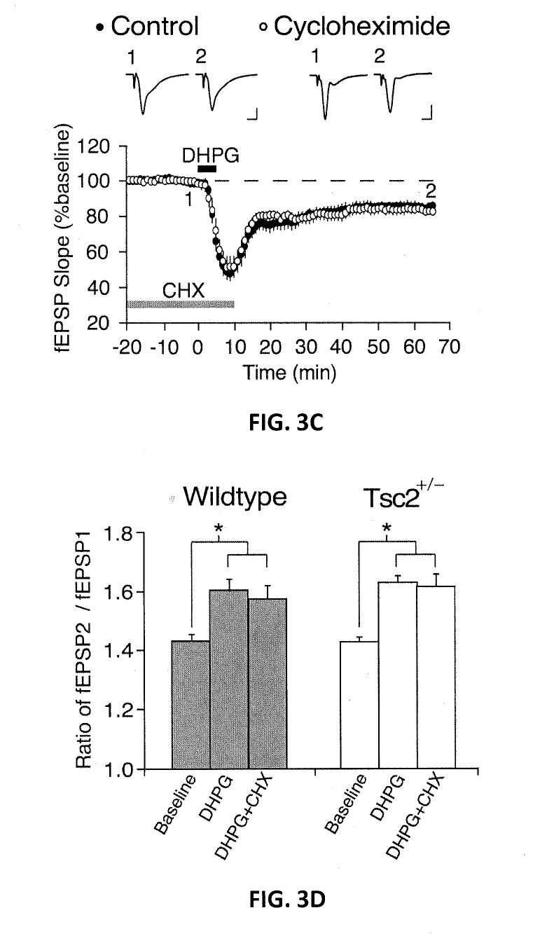

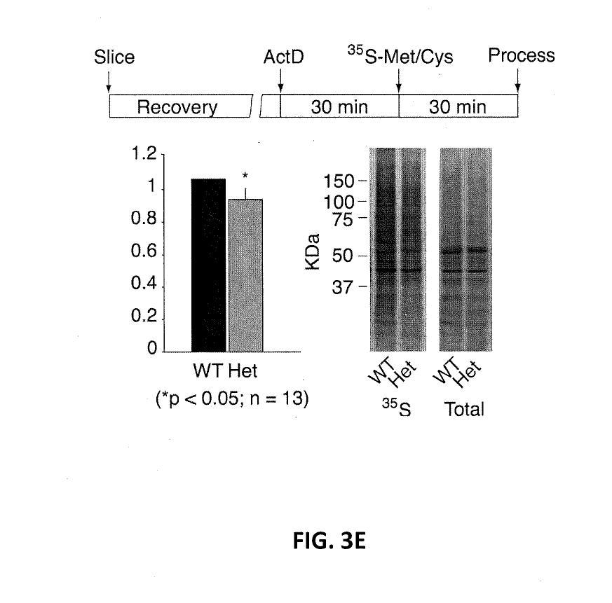

LTD in WT mice (C57B1/6J) at the age range examined (postnatal day (P) 25-30) was reliably reduced by the protein synthesis inhibitor cycloheximide (60 μM; FIG. 3A). The presynaptic component of LTD was monitored by measuring paired-pulse facilitation (PPF), which showed a persistent increase following DHPG (FIG. 3D) that has been ascribed to reduced probability of glutamate released by the first pulse (Fitzjohn, S. M., et al., J. Physiol. 537: 421 (2001); and Nosyreva, E. D., et al., J. Neuroscience 25: 2992 (2005)). Changes in PPF are not inhibited by cycloheximide (FIG. 3D), suggesting that residual LTD in the presence of the drug is expressed presynaptically.

FIG. 3A shows that application of the Gp1mGluR selective agonist R,S-DHPG (50 μM) or S-DHPG (25 μM) for 5 min (black bar) induces LTD in area CA1 of hippocampal slices from WT mice. LTD is significantly attenuated by pretreatment with the protein synthesis inhibitor cycloheximide (CHX, 60 μM, gray bar) (control: 76±2.6%, n=11; CHX: 85.6±3.4%, n=7; *p<0.01). FIG. 3B shows that DHPG induces significantly less LTD in slices from Tsc2^{+/-} mice as compared to slices from littermate WT mice (WT: 74.1±2.0%, n=10; Tsc2^{+/-} 85.2±2.7%, n=12; *p<0.01). FIG. 3C shows that CHX treatment has no effect on DHPG-LTD in slices from Tsc2^{+/-} mice (control: 85.2±2.7%, n=12; CHX: 83.5±2.1%, n=7, p=0.61). Representative field potential traces (average of 10 sweeps) were taken at times indicated by numerals. Scale bars equal 0.5 mV, 5 ms in FIG. 3D, which shows presynaptic LTD is not affected by genotype or CHX. PPF was assessed during the baseline period and 60 minutes post DHPG application in slices either pretreated with CHX or control ACSF. DHPG significantly increased PPF in slices from both wildtype and Tsc2^{+/-} mice (PPF with a 50 ms inter-stimulus interval: WT baseline: 1.43±0.02, WT DHPG: 1.59±0.04, n=9, *p<0.001; Tsc2^{+/-} baseline: 1.43±0.02, Tsc2^{+/-} DHPG: 1.63±0.02, n=9, *p<0.001) and this effect was not blocked by cycloheximide (WT DHPG+CHX: 1.58±0.05, n=11, p=0.84; Tsc2^{+/-} DHPG+CHX: 1.62±0.04, n=7, p=0.80).

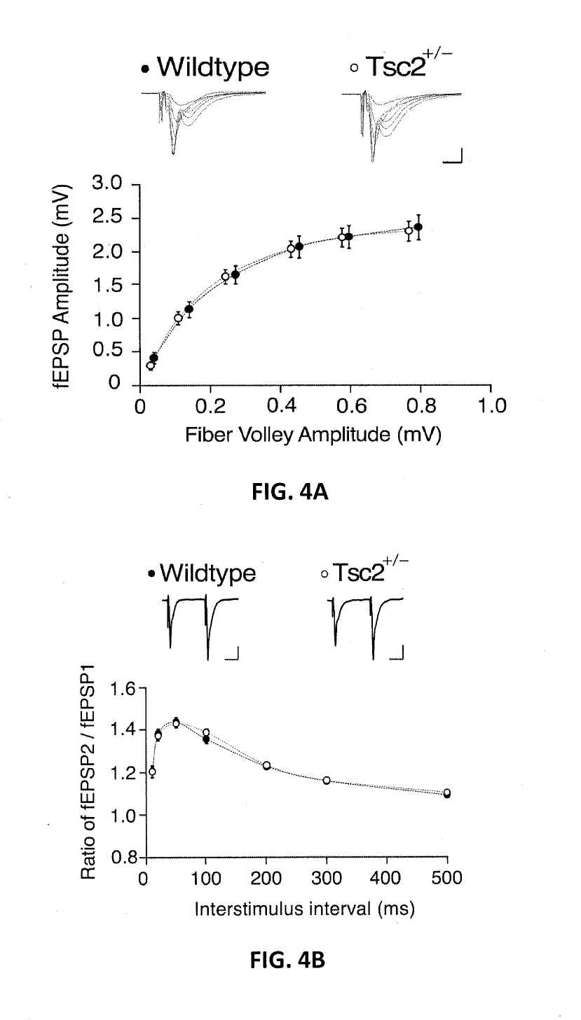

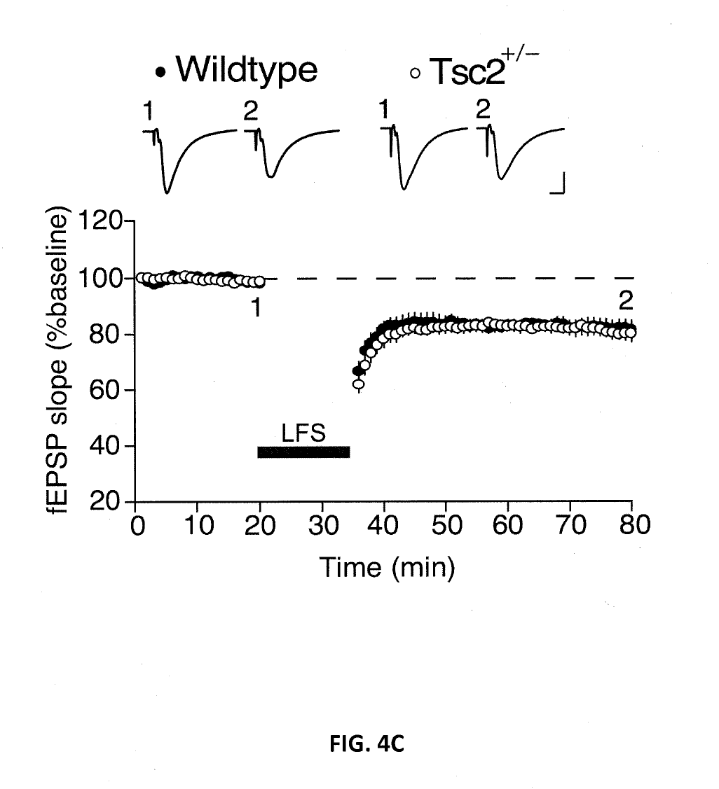

Basal synaptic transmission in CA1 appears to be normal in the Tsc2^{+/-} mice and there is no difference in the NMDA receptor-dependent form of LTD (FIGS. 4A-4C), demonstrating there is a specific deficit in mGluR-dependent LTD in the Tsc2^{+/-} mutants (FIG. 3B). The persistent PPF change after DHPG was no different in the mutants than in WT, however, suggesting a deficient postsynaptic modification (FIG. 3D). Unlike WT, cycloheximide treatment had no effect on LTD in the Tsc2^{+/-} animals (FIG. 3C). These data suggest a selective loss of the protein synthesis-dependent component of LTD in the mutant mice. Therefore, protein synthesis rates were directly measured in slices from wildtype and Tsc2^{+/-} mice. FIG. 3E shows that hippocampal slices from Tsc2^{+/-} mice have decreased protein synthesis rates compared to wild type controls (WT: 100±3%; TSC: 88.2±3%; n=12, p<0.05), suggesting that elevated mTOR signaling may lead to decreased protein synthesis, resulting in the loss of the protein synthesis-dependent component of LTD.

FIG. 4A shows basal synaptic transmission (plotted as fEPSP amplitude against presynaptic fiber volley amplitude) does not differ between genotypes. Scale bars equal 0.5 mV, 5 ms for representative field potential traces. FIG. 4B shows paired pulse facilitation is normal across several inter-stimulus intervals in Tsc2^{+/-} mice. Scale bars equal 0.5 mV, 20 ms representative field potential traces. FIG. 4C shows the magnitude of NMDAR dependent LTD evoked by low frequency stimulation (LFS, 900 pulses at 1 Hz) does not differ between genotypes (WT: 78.9±3.8%, n=6; Tsc: 77.1±2.7%, n=6; p=0.69). Representative field potential traces (average of 10 sweeps) were taken at times indicated by numerals. Scale bars equal 0.5 mV, 5 ms.

mGluR-long term depression (LTD) is a well characterized Gp1 mGluR mediated processes. Activation of Gp1 mGluR can have a myriad of cellular and synaptic effects (Lee, A. C., et al., J Neurophysiol 88, 1625-33 (2002); Vanderklish, P. W., et al., Proc Natl Acad Sci USA 99, 1639-44 (2002); Neyman, S., et al., Eur J Neurosci 27, 1345-52 (2008); and Francesconi, W., et al., Brain Res 1022, 12-8 (2004)). Many of these changes are dependent upon rapid, de novo protein synthesis (Merlin, L. R., et al., J Neurophysiol 80, 989-93 (1998); and Raymond, C. R., et al., J Neurosci 20, 969-76 (2000)). It is likely that most of these processes contribute to the multiple symptoms seen in TSC, which includes mGluR-LTD. There is an established correlation between the level of mGluR-LTD and protein synthesis rates in the hippocampus, and regulating mGluR5 activity has been shown to have a direct effect on protein synthesis rates (Dolen, G., et al., Neuron 56, 955-62 (2007)). As described herein, there is deficient mGluR dependent LTD and protein synthesis in Tsc2^{+/-} mice. The deficiency in mGluR-dependent LTD can be rescued (also referred to herein as “normalized” or “restored”) by acute mTOR inhibition, suggesting this deficiency is a result of elevated mTOR signaling. As also described herein, increasing mGluR5 activity with mGluR5 PAM treatment can restore mGluR-LTD in a protein synthesis dependent manner.

Effect of Rapamycin and mGluR5 PAM Treatment on mGluR-LTD Rates in Tsc2^{+/-} Mice

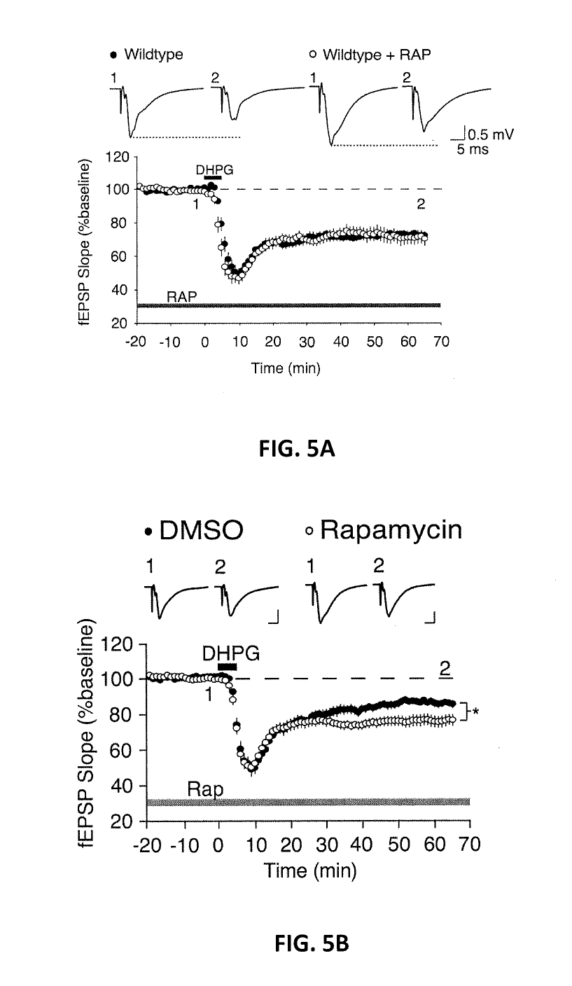

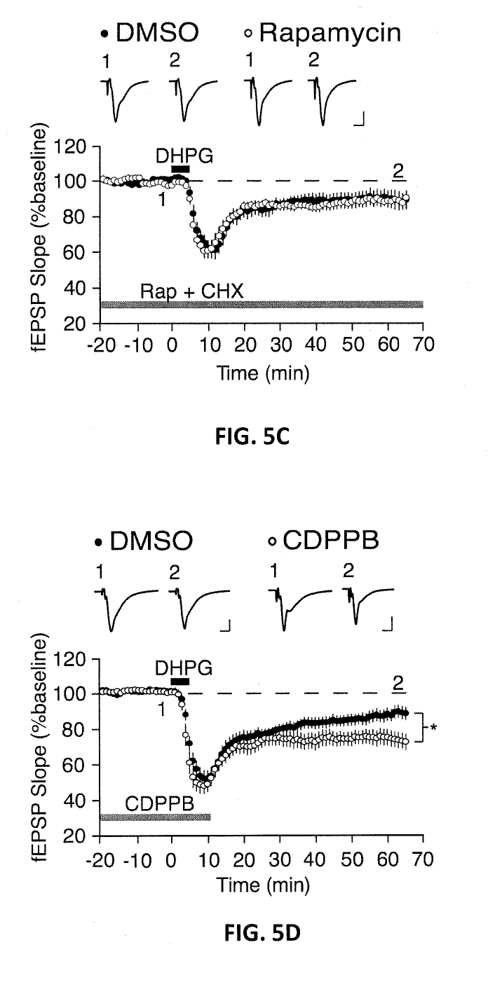

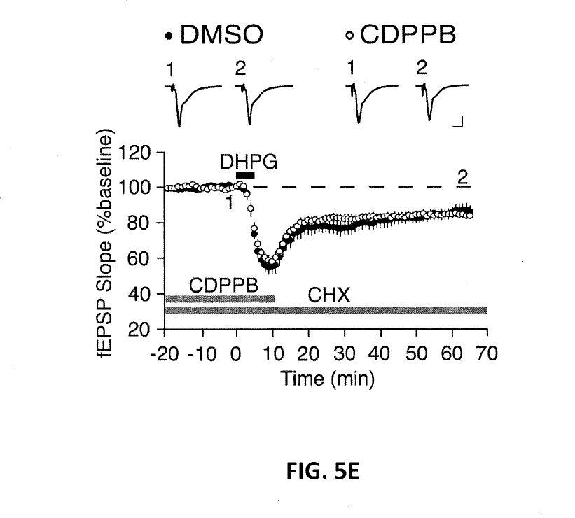

As in the human disease, the germ line mutation in TSC2 can have myriad secondary consequences on neural development that could contribute to the observed LTD phenotype. To determine if deficient LTD is a specific consequence of unregulated mTOR activity, the effects of the mTORC1 inhibitor rapamycin (20 nM) was evaluated. Pretreatment of slices with rapamycin (RAP, 20 nM, gray bar) has no effect on DHPG (FIG. 9A, 50 μM, black bar) induced LTD in hippocampal slices from wildtype animals. However, acute rapamycin treatment in slices from Tsc2^{+/-} mice normalized LTD to the WT level (FIG. 5B). This rescue of LTD is due specifically to the recovery of the protein synthesis-dependent component, as the effect of rapamycin in the Tsc2^{+/-} mice was eliminated in the presence of cycloheximide (FIG. 5C). Thus, unregulated mTOR activity in the Tsc2^{+/-} mice appears to suppress the synaptic protein synthesis required for mGluR-LTD.

In the Fmr1 KO model of fragile X syndrome (Huber, K. M., et al., Proc. Natl. Acad. Sci. USA 99: 7746-7750 (2002)), excessive mGluR-LTD and hippocampal protein synthesis, as shown herein, can be corrected by reducing signaling by mGluR5. Pretreatment of hippocampal slices with the mGluR5 positive allosteric modulator (PAM) 3-Cyano-N-(1,3-diphenyl-1H-pyrazol-5-yl)benzamide (CDPPB⁽⁶⁰⁾) restored the magnitude of mGluR-LTD in Tsc2^{+/-} mice to WT levels (FIG. 5D). The rescue of LTD appears to be due specifically to recovery of the protein synthesis-dependent component because the effect of CDPPB was completely eliminated by cycloheximide (FIG. 5E). Thus, allosteric augmentation of mGluR5 signaling can overcome the inhibitory effect of unregulated mTOR activity on the synaptic protein synthesis that supports LTD.

FIG. 5A shows that pretreatment of slices with the mTORC1 inhibitor rapamycin (RAP, 20 nM, gray bar) has no effect on DHPG induced LTD in slices from wildtype animals (DMSO: 71.4% n=12; RAP: 73.0%, n=12; p>0.6). FIG. 5B shows that pretreatment of slices with rapamycin significantly enhances DHPG induced LTD in slices from Tsc2^{+/-} mice (DMSO: 85.4±2.2%, n=13; RAP: 75.3±3%, n=14; *p<0.02). FIG. 5C shows the effect of rapamycin on DHPG induced LTD in Tsc2^{+/-} mice is prevented by the protein synthesis inhibitor cycloheximide (DMSO: 89.5±5.1%, n=7; RAP: 89.3±2.4%, n=8; p=0.99). FIG. 5D shows the pretreatment of slices from Tsc2^{+/-} mice with the mGluR5 positive allosteric modulator CDPPB (10 μM, gray bar) significantly enhances DHPG induced LTD (control: 87.6±3.4%, n=12; CDPPB: 72.7±4.4%, n=10; *p<0.02). FIG. 5E shows that CDPPB treatment fails to enhance DHPG induced LTD in Tsc2^{+/-} mice when co-applied with the protein synthesis inhibitor cycloheximide (DMSO: 87.1±3.5% n=9; CDPPB: 84.8±1.8%, n=9; p=0.65). Representative field potential traces (average of 10 sweeps) were taken at times indicated by numerals. Scale bars equal 0.5 mV, 5 ms.

Altered mGluR5 signaling may be associated with the cognitive deficits in TSC and restoring proper mGluR5 function may alleviate these deficits. Deficient mGluR-LTD was restored in Tsc2^{+/-} mice with a positive allosteric modulator (PAM) of mGluR5. mGluR PAMs (e.g., mGluR5PAMs) are compounds that do not activate mGluR5 directly, but act on an allosteric site to potentiate physiological activation of the receptor by its natural ligand glutamate or a synthetic agonist, such as DHPG. Because mGluR PAMs do not directly activate or inhibit mGluR activity, but rather modulate the receptor response to endogenous activation, mGluR5PAMs have the attribute of enhancing mGluR5 activity in a physiologically relevant way. Pretreatment of hippocampal slices with the mGluR5CDPPB significantly enhanced the level of mGluR-LTD seen in Tsc2^{+/-} mice to levels comparable with wildtype animals.

In the presence of protein synthesis inhibitors, PAM treatment no longer has an effect on mGluR-LTD. This demonstrates that PAM treatment restores mGluR-stimulated protein synthesis in Tsc2^{+/-} mice, which is reflected in the enhancement of mGluR-LTD to thereby normalize LTD. These data show that positive allosteric modulation of mGluR5 rescue the electrophysiological deficits in Tsc2^{+/-} mice in a protein synthesis dependent manner. Thus, mGluR5 PAM treatment may be a viable therapy for the cognitive impairments associated with an elevation in mTOR signaling, such as TSC. Rapamycin treatment is a pharmacological rescue of TSC because it reconstitutes the negative regulation of mTOR normally imposed by the TSC1/TSC2 complex. While rapamycin has been used clinically, it is not an ideal drug for the chronic treatment of TSC due to its strong immunosuppressive properties. Direct modulation of mGluR activity with the use of PAMs may be an effective therapeutic strategy for the treatment of TSC that has minimal side-effects.

### Example 2

### Treatment with mGluR5 PAM

These data show that there is disrupted mGluR function in the hippocampus of Tsc2^{+/-} mice. The hippocampus is an area of the brain known to be vital for many forms of learning and memory (Eichenbaum, H., et al., Neuron 44, 109-20 (2004)). Alterations in hippocampal function have adverse effects on learning and cognition, and therefore are likely to contribute to the cognitive impairments seen in TSC. As shown herein, mGluR5 PAM and rapamycin treatment reverse the electrophysiological impairments in the hippocampus of Tsc2^{+/-} mice. Rapamycin treatment has been shown to reverse the deficits in hippocampal-dependent learning observed in these mice. Therefore, mGluR5 PAM treatment may also reverse hippocampal-dependent deficiencies in Tsc2^{+/-} mice. The nature of the relationship between mTOR signaling, mGluR dependent plasticity, and the electrophysiological and behavioral impairments seen in Tsc2^{+/-} mice can be further evaluated by examining the effect of mGluR5 PAM treatment on the behavioral impairments previously shown to be rescued by rapamycin in Tsc2^{+/-} mice, as described infra.

Context fear-conditioning experiments were employed to assess improvements in cognitive processing following administration of a composition that activates Group I mGluR signaling, such as by administration of mGluR5PAMs. Context fear-conditioning was performed using a modification of a previously described procedure (Ehninger, D., et al., Nat Med 14, 843-8 (2008). Wildtype and Tsc2^{+/-} mice (8-12 weeks old) are habituated to the testing room and experimenter for 3 days prior to training On the day of training, mice is placed in the training context and delivered one 0.80 mA shocks (2 sec). The mice were allowed about 3 minutes to explore context before conditioning and were removed about 15 sec after the shock was given and returned to home cage. Conditioned fear response was assessed 24 hours later by a trained observer measuring the percentage of time spent freezing during the test period (about 3 min session). To determine context specificity of the conditioned response, mice trained at the same time were separated into two groups: one group was tested in the same training context and the other tested in a novel context. This novel context was created by varying: distal cues, odor, floor material, and lighting of the testing apparatus. For rescue experiments, animals received a single injection of CDPPB (10 mg/kg, i.p.) about 30 minutes prior to training session.

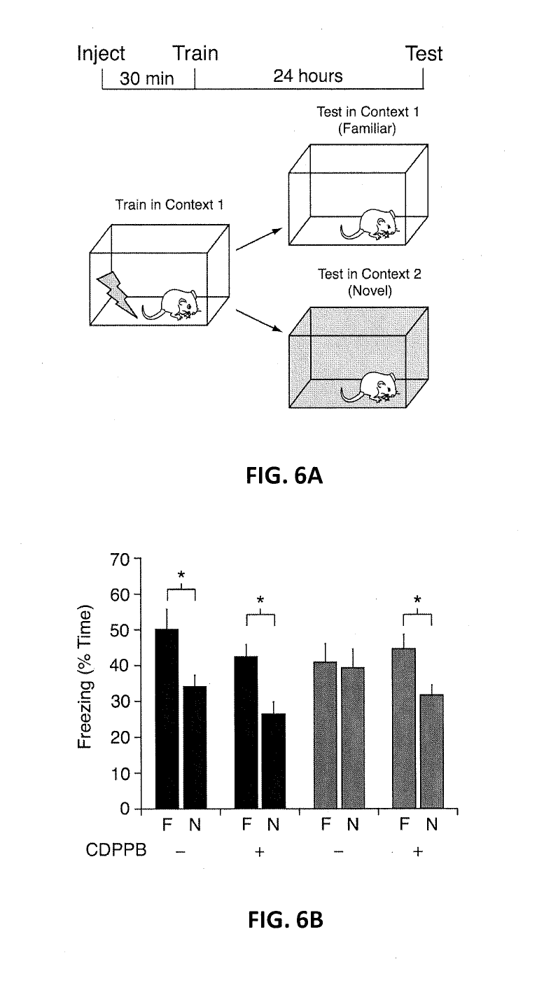

Cognitive impairments in the Tsc2^{+/-} mice were improved by treating the animals with the mTORC1 inhibitor rapamycin (Ehninger, D., et al., Nat. Med. 14: 843-848 (2008)). A robust phenotype was reported to be an impairment in the ability of the Tsc2^{+/-} mice to distinguish between familiar and novel contexts in a fear conditioning paradigm. The advantage of this paradigm is the learning occurs in one trial, making it amenable to acute drug treatment, and the memory is hippocampus-dependent. The mice are first exposed to a distinctive context in which they receive an aversive foot shock. The next day, context discrimination is tested by dividing the animals into two groups, one placed in the familiar context associated with the shock, and the other placed in a novel context (FIG. 6A). Context discrimination is assessed by measuring the time the animals express fear by freezing in each context. Although the WT mice clearly discriminate between contexts, the Tsc2^{+/-} mice do not (FIG. 6B). To test the effect of augmenting mGluR5 signaling, mice from both genotypes were injected i.p. with CDPPB (10 mg/kg) 30 minutes prior to training Although this treatment had no effect in the WT mice, it was sufficient to correct the deficit in context discrimination observed in the Tsc2^{+/-} mice (FIG. 6B).

As shown in FIG. 6A, memory of the context in which the shock was received (context 1) was assessed 24 hours later by comparing freezing of one cohort of trained animals in the familiar context (context 1) with freezing of a second cohort in a novel context (context 2). FIG. 6B shows wildtype (WT) mice display intact memory by freezing more in the familiar context (F) than the novel context (N) (Black bars; Familiar: 50±7.7%, n=12; Novel: 34.1±3.2%, n=14; *p<0.01). A single injection of CDPPB (10 mg/kg, i.p.) 30 minutes prior to training has no effect on WT context discrimination (Familiar: 42.3±3.7%, n=12; Novel: 26.4±3.6%, n=12; *p<0.01). Control Tsc2^{+/-} mice display a significant impairment in context discrimination (Red bars; Familiar: 40.9±5.3%, n=11; Novel: 39.3±5.2%, n=14; p=0.83), but this deficit is corrected by a single injection of CDPPB (Familiar: 44.5±4.3%, n=11; Novel: 31.6±3%, n=12; *p<0.05).

These data are important to further understand the mechanism of synaptic protein synthesis and LTD triggered by mGluR5 and for designing therapeutic treatment of cognitive impairments associated with an elevation in mTOR signaling, such as those observed in TSC. In the fragile X knock out mouse model, basal protein synthesis is elevated and LTD is exaggerated downstream of an mGluR5 signaling pathway, which appears to involve the mitogen activated kinase ERK1/2 Inhibition of mGluR5 corrects aspects of fragile X syndrome in animal models. Recent data suggest that the mTOR signaling pathway is also constitutively overactive in the Fmr1 KO mouse (Sharma, A., et al., J. Neurosci. 30: 694 (2010)), however, the relevance to exaggerated protein synthesis and altered synaptic function is controversial. The current findings show that inhibition of mTOR signaling with rapamycin rescues LTD in the Tsc2^{+/-} mice, suggesting that increased synaptic mTOR activity suppresses the protein synthesis required for LTD in these animals (FIGS. 2, 7 and 8). Precisely how excess mTOR activity suppresses synthesis of “LTD proteins” may be due to hyperphosphorylation of FMRP, or increased translation of a competing pool mRNAs unrelated to LTD.

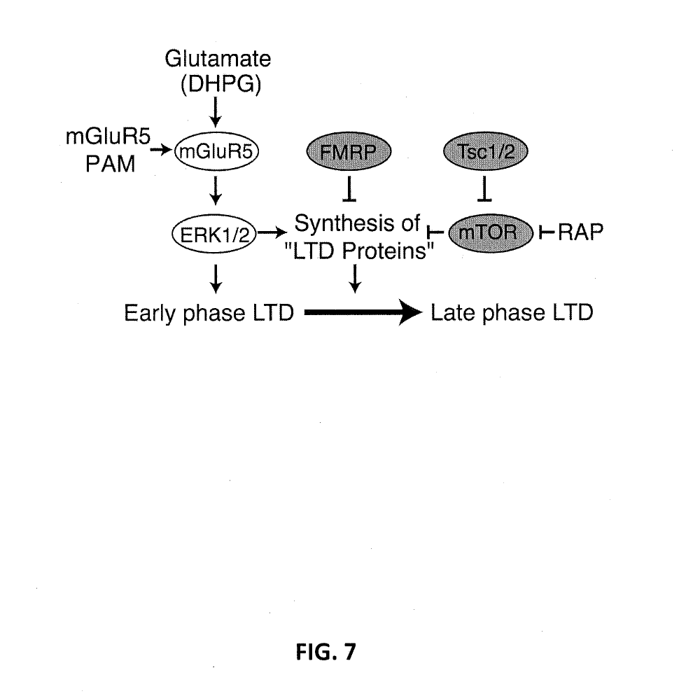

Activation of mGluR5 by glutamate or DHPG rapidly triggers synaptic depression that is stabilized by a process that normally requires immediate translation of synaptically localized mRNAs (FIG. 7). De-repression of mTOR at the synapse impairs the protein synthesis required for LTD. This impairment can be overcome either by inhibiting mTOR with rapamycin or by augmenting mGluR5 signaling with the PAM (FIG. 7). Signaling by mGluR5 appears to be a critical regulator of local mRNA translation. In fragile X syndrome, impaired functions caused by excessive local protein synthesis can be corrected by a negative allosteric modulator (NAM) of mGluR5. In conditions characterized by an elevation in mTOR signaling, such as TSC, impaired functions (e.g., cognitive impairment) caused by reduced local protein synthesis are restored by an mGluR5 PAM, as shown herein.

The current findings are also relevant for the treatment of behavioral deficits associated with an elevation in mTOR signaling in conditions such as TSC. Previous studies in the Tsc2^{+/-} mouse raised the possibility that cognitive aspects of TSC may be ameliorated with rapamycin, even when treatment starts in adulthood (Ehninger, D., et al., Nat. Med. 14: 843-848 (2008)). As described herein, mGluR PAMs, such as mGluR5 PAM, may be similarly effective. While rapamycin has been used clinically, it is problematic for chronic treatment because of its strong immunosuppressive properties. An advantage of treatment by compounds that activate Group I mGluR signaling, such as mGluR5PAMs, is that they specifically target the synaptic mechanisms that are likely responsible for the cognitive and behavioral impairments in TSC rather than having overall systematic effects.

Unlike, the Fmr1 mutation that results in fragile X syndrome, the Tsc2 mutation causes diminished synaptic protein synthesis and LTD that are corrected by augmentation of mGluR5 (FIGS. 7 and 8). Gain- and loss-of-function mutations in individual genes, such as MECP2, may result in syndromes with overlapping features, such as epilepsy, cognitive impairment and autism spectrum disorder. However, the mechanism that results in such features does not appear to be similar or universal.

The teachings of all of the references cited herein are hereby incorporated by reference in their entirety.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a subject having elevated signaling of a mammalian target of rapamycin (mTOR), comprising the step of administering to the subject a composition that includes at least one compound that activates Group I mGluR signaling.

2. The method of claim 1, wherein the subject further has at least one condition selected from the group consisting of mental retardation and autism.

3. The method of claim 2, wherein the subject further has tuberous sclerosis complex.

4. The method of claim 2, wherein the subject further has PTEN hamartoma syndrome.

5. The method of claim 1, wherein the compound includes a Group I mGluR agonist.

6. The method of claim 1, wherein the compound includes a Group I mGluR positive allosteric modulator.

7. The method of claim 1, wherein the compound activates Group I mGluR5 signaling.

8. The method of claim 1, wherein the compound activates Group I mGluR1 signaling.

9. The method of claim 1, wherein the compound is administered to the subject in a single daily dose.

10. The method of claim 1, wherein the compound is administered to the subject in multiple daily doses.

11. The method of claim 1, wherein the subject has at least one impairment in a cognitive function.

12. The method of claim 11, wherein the impairment is selected from the group consisting of a memory impairment, an executive function impairment and a speed of processing impairment.

13. The method of claim 1, wherein the subject has a seizure disorder.

14. The method of claim 13, wherein the seizure disorder includes at least one member selected from the group consisting of an audiogenic seizure and an epileptic seizure.
